(19) United States
(12) Patent Application Publication
(10) Pub. No.: US 2009/0318422 A1
(43) Pub. Date: Dec. 24, 2009

(54) OPHTHALMIC PERCUTANEOUS ABSORPTION TYPE PREPARATION

(76) Inventors: Akiharu Isowaki, Hyogo (JP); Tomoko Nakajima, Hyogo (JP); Akira Ohtori, Hyogo (JP)

Correspondence Address: WENDEROTH, LIND & PONACK, L.L.P., 1030 15th Street, N.W.,, Suite 400 East, Washington, DC, 20005-1503, US

(21) Appl. No.: 12/310,281

(22) PCT Filed: Aug. 28, 2007
(86) PCT No.: PCT/JP2007/067103

§ 371 Date: Mar. 11, 2009

Related U.S. Application Data
(60) Provisional application No. 60/840,462, filed on Aug. 28, 2006.

(51) Int. Cl. A61K 31/55; A61K 31/44; A61K 31/335

(52) U.S. Cl. 514/214.02; 514/324; 514/450

(57) ABSTRACT

The present invention provides an ophthalmic percutaneous absorption type preparation containing an ophthalmic drug and a vasoconstrictor, which can increase the amount of the ophthalmic drug transferred through the eyelid to a topical area in the eye, particularly the anterior segment of the eye such as conjunctiva, lacrimal fluid, aqueous humor, cornea and the like by administration to the skin surface of an eyelid.

### TECHNICAL FIELD

The present invention relates to an ophthalmic percutaneous absorption type preparation that increases, when an ophthalmic drug is administered to the skin surface of an eyelid, the amount of transfer of the ophthalmic drug to a topical area in the eye through the eyelid, and a method of increasing the amount of transfer of the ophthalmic drug to a topical area in the eye through the eyelid.

### BACKGROUND ART

As a conventional form of an ophthalmic pharmaceutical agent to be topically applied to the eye, eye drop is most generally adopted. Eye drop, however, shows low ocular topical bioavailability of the drug due to the influence of turnover of the lacrimal fluid on the surface of the eye, where long duration of efficacy sometimes requires frequent instillation.

Recently, as one of the preparations for the treatment of ophthalmic diseases, a percutaneous absorption type preparation for the treatment of an ophthalmic disease has been proposed, which has a structure wherein a plaster layer containing a therapeutic drug for the ophthalmic disease is formed on a support, which is to be adhered to the skin surface including the outside surface of an eyelid to allow administration of the therapeutic drug for the ophthalmic disease in the plaster layer to a topical tissue in the eye substantially without via the systemic blood flow but through the skin (WO2004/064817 and US2006/0036220A1).

Generally, a transdermally administered drug is transferred from the surface layer to epidermis, dermis, subcutaneous tissue, muscle and the like. Since most of the administered drug is delivered to the subcutaneous blood vessel network in the dermis and transported to the systemic circulatory system, the amount of topically transferred drug is considered to be very small.

To improve transferability to the muscle, subcutaneous tissue and the like, therefore, a method comprising concurrent use of a vasoconstrictor to suppress uptake of the drug into the blood flow has been reported (Int. J. Pharm. 288 (2005) 227-233; J. Pharm. Sci. 83 (1994) 783-791).

However, the above-mentioned references do not describe a method of increasing the transfer amount of an ophthalmic drug into the topical area in the eye, particularly, anterior segment of the eye, by administration of an ophthalmic percutaneous absorption preparation containing a vasoconstrictor to the skin surface of an eyelid, and such an ophthalmic percutaneous absorption preparation.

### DISCLOSURE OF THE INVENTION

It is therefore an object of the present invention to provide an ophthalmic percutaneous absorption type preparation capable of increasing the transfer amount of an ophthalmic drug into the anterior segment of the eye such as topical area in the eye, particularly conjunctiva, lacrimal fluid, aqueous humor, cornea and the like, through an eyelid, by administering the ophthalmic percutaneous absorption type preparation containing the ophthalmic drug to the skin surface of the eyelid.

It is another object of the present invention to provide a method of increasing the transfer amount of an ophthalmic drug into the anterior segment of the eye through an eyelid, by administering the ophthalmic percutaneous absorption type preparation containing the ophthalmic drug to the skin surface of the eyelid.

The present inventors have found that a transfer amount of an ophthalmic drug, particularly an antiallergic agent, into a topical area in the eye, particularly the anterior segment of the eye (e.g., conjunctiva, lacrimal fluid, aqueous humor, cornea and the like), can be increased through an eyelid by a combined use of an ophthalmic drug and a vasoconstrictor, for example, by administering a preparation obtained by adding a vasoconstrictor to an ophthalmic percutaneous absorption type preparation comprising an ophthalmic drug to the skin surface of the eyelid, which resulted in the completion of the present invention.

Accordingly, the present invention relates to the following.

(1) An ophthalmic percutaneous absorption type preparation comprising an ophthalmic drug and a vasoconstrictor in combination.
(2) An ophthalmic percutaneous absorption type preparation comprising an ophthalmic drug and a vasoconstrictor.
(3) The ophthalmic percutaneous absorption type preparation of the above-mentioned (1) or (2), which is administered to the skin surface of an eyelid.
(4) The ophthalmic percutaneous absorption type preparation of any of the above-mentioned (1) to (3), wherein the ophthalmic drug is an agent for the prophylaxis or treatment of a disease in the anterior segment of the eye.
(5) The ophthalmic percutaneous absorption type preparation of the above-mentioned (4) wherein the agent for the prophylaxis or treatment of a disease in the anterior segment of the eye is at least one selected from an antiallergic agent, a therapeutic agent for dry eye, an anti-inflammatory agent, an antibacterial agent and an antiglaucoma agent.
(6) The ophthalmic percutaneous absorption type preparation of the above-mentioned (5), wherein the agent for the prophylaxis or treatment of a disease in the anterior segment of the eye is an antiallergic agent.
(7) The ophthalmic percutaneous absorption type preparation of the above-mentioned (6), wherein the antiallergic agent is at least one selected from ketotifen, olopatadine, epinastine and a pharmaceutically acceptable salt thereof.
(8) The ophthalmic percutaneous absorption type preparation of the above-mentioned (7), wherein the antiallergic agent is ketotifen fumarate or olopatadine hydrochloride.
(9) The ophthalmic percutaneous absorption type preparation of any of the above-mentioned (1) to (8), wherein the vasoconstrictor is phenylephrine hydrochloride.
(10) A method of increasing the amount of transfer of an ophthalmic drug to a topical area in the eye through the eyelid, which comprises a step of administering an ophthalmic drug to the skin surface of an eyelid under the conditions where a vasoconstrictor is present from the skin surface of an eyelid to the inside of the eyelid and/or conjunctiva.
(11) The method of the above-mentioned (10), wherein the topical area in the eye is the anterior segment of the eye.
(12) The method of the above-mentioned (10), wherein the ophthalmic drug is an agent for the prophylaxis or treatment of a disease in the anterior segment of the eye.
(13) The method of the above-mentioned (12), wherein the agent for the prophylaxis or treatment of the disease in the anterior segment of the eye is at least one selected from an antiallergic agent, a therapeutic agent for dry eye, an anti-inflammatory agent, an antibacterial agent and an antiglaucoma agent.
(14) The method of the above-mentioned (13), wherein the agent for the prophylaxis or treatment of the disease in the anterior segment of the eye is antiallergic agent.
(15) The method of the above-mentioned (14), wherein the antiallergic agent is at least one selected from ketotifen, olopatadine, epinastine and a pharmaceutically acceptable salt thereof.
(16) The method of the above-mentioned (15), wherein the antiallergic agent is ketotifen fumarate or olopatadine hydrochloride.
(17) The method of any of the above-mentioned (10)-(16), wherein the vasoconstrictor is phenylephrine hydrochloride.
(18) A method of treating an ophthalmic disease, which comprises a step of administering an effective amount of an ophthalmic drug and a vasoconstrictor to a subject of administration in need of the treatment.
(19) Use of an ophthalmic drug and a vasoconstrictor for the production of an ophthalmic percutaneous absorption type preparation containing an ophthalmic drug and a vasoconstrictor.

### BEST MODE FOR EMBODYING THE INVENTION

The ophthalmic percutaneous absorption preparation of the present invention is a preparation containing an ophthalmic drug and a vasoconstrictor in combination, and may be any as long as the ophthalmic drug and the vasoconstrictor can be combined on administration (hereinafter sometimes to be referred to as the preparation of the present invention).

Accordingly, as long as an ophthalmic drug and a vasoconstrictor can be combined on administration, the preparation of the present invention may be a single preparation obtained by simultaneously formulating an ophthalmic drug and a vasoconstrictor, or a combination of two kinds of preparations obtained by separately formulating an ophthalmic drug and a vasoconstrictor.

A preferable preparation of the present invention is a preparation containing an ophthalmic drug and a vasoconstrictor, i.e., a single preparation obtained by simultaneously formulating an ophthalmic drug and a vasoconstrictor.

The administration mode is not particularly limited as long as an ophthalmic drug is administered to the skin surface of an eyelid under the conditions where a vasoconstrictor is present from the skin surface of an eyelid to the inside of the eyelid and/or conjunctiva and, for example, (1) administration of a composition containing an ophthalmic drug and a vasoconstrictor, namely, administration as a single preparation, (2) simultaneous administration of two kinds of preparations obtained by separately formulating an ophthalmic drug and a vasoconstrictor, (3) administration of two kinds of preparations of an ophthalmic drug and a vasoconstrictor, which have been separately formulated, by the same administration route in a time staggered manner (for example, administration in the order of the vasoconstrictor and the ophthalmic drug, or in the reverse order), (4) simultaneous administration of two different kinds of preparations of an ophthalmic drug and a vasoconstrictor, which have been separately formulated (for example, gel preparation and adhesive preparation and the like), (5) administration of two kinds of preparations of an ophthalmic drug and a vasoconstrictor, which have been separately produced, by different administration routes in a time staggered manner (for example, administration in the order of gel vasoconstrictor preparation and adhesive preparation of ophthalmic drug, and the like) and the like can be mentioned. For example, when the absorption of the ophthalmic drug is fast and the absorption of the vasoconstrictor is slow, the effect of the ophthalmic drug is enhanced by administering the vasoconstrictor in advance.

In the preparation of the present invention, the combination ratio of an ophthalmic drug and a vasoconstrictor is generally within the range of 1:0.001-10, preferably within the range of 1:0.005-5, and more preferably within the range of 1:0.01-5, in weight ratio, whether they are processed into a single preparation or independent preparations.

When an antiallergic agent is used as an ophthalmic drug, for example, a combination ratio of an antiallergic agent and a vasoconstrictor is generally within the range of 1:0.001-10, preferably within the range of 1:0.005-5, and more preferably within the range of 1:0.01-5, in weight ratio. When an antiallergic agent is used as an adhesive preparation, a combination ratio of an antiallergic agent and a vasoconstrictor is generally within the range of 1:0.001-10, preferably within the range of 1:0.005-5, and more preferably within the range of 1:0.01-5, in weight ratio. For use as an ointment or gel preparation, a combination ratio of an antiallergic agent and a vasoconstrictor is generally within the range of 1:0.001-10, preferably within the range of 1:0.005-5, and more preferably within the range of 1:0.01-5, in weight ratio.

The ophthalmic drug in the present invention includes any pharmaceutical agent used for the prophylaxis or treatment of ophthalmic diseases, and includes a surgical agent, a test agent and the like. Preferably, it is an agent for the prophylaxis or treatment of a disease in the anterior segment of the eye. Examples of the disease in the anterior segment of the eye include allergic conjunctivitis, vernal keratoconjunctivitis, contact eyelid conjunctivitis, phlyctenular keratoconjunctivitis, giant papillary conjunctivitis, atopic keratoconjunctivitis, pollinosis, dacryocystitis, dry eye, Sjogren's syndrome, Stevens-Johnson syndrome, meibomianitis, hypolacrimia, hordeolum, blepharitis, keratitis, corneal ulcer, eye infection, glaucoma and the like.

Examples of an agent for the prophylaxis or treatment of such diseases in the anterior segment of the eye include antiallergic agent, therapeutic agent for dry eye, anti-inflammatory agent, antibacterial agent, antiglaucoma agent and the like. Preferred is antiallergic agent. Examples of the target disease of antiallergic agent include allergic conjunctivitis, vernal keratoconjunctivitis, contact eyelid conjunctivitis, phlyctenular keratoconjunctivitis, giant papillary conjunctivitis, atopic keratoconjunctivitis, pollinosis and the like.

The antiallergic agent in the present invention may be any as long as it has an antiallergic action and includes ketotifen, olopatadine, epinastine, azelastine, diphenhydramine, levocabastine, tranilast, amlexanox, pemirolast potassium, ibudilast, acitazanolast, fexofenadine, cetirizine, loratadine, cyproheptadine, promethazine or a pharmaceutically acceptable salt thereof, cyclosporine, sodium cromoglycate, chlorpheniramine maleate and the like can be mentioned. Preferred is ketotifen, olopatadine, epinastine or a pharmaceutically acceptable salt thereof. More preferred are ketotifen fumarate and olopatadine hydrochloride.

Examples of the therapeutic agent for dry eye include pilocarpine, cevimeline, carbachol, cyclosporine, rebamipide, rimexolone, pimecrolimus, a pharmaceutically acceptable salt thereof and the like.

Examples of the anti-inflammatory agent include bromfenac, pranoprofen, diclofenac, ketorolac, amfenac, nepafenac, indomethacin, dexamethasone, betamethasone, fluorometholone, loteprednol, difluprednate, prednisolone, a pharmaceutically acceptable salt thereof and the like.

Examples of the antibacterial agent include lomefloxacin, norfloxin, enoxacin, ofloxacin, ciprofloxacin, tosufloxacin, fleroxacin, cinoxacin, levofloxacin, sparfloxacin, moxifloxacin, trovafloxacin, azithromycin, clarithromycin, cefdinir, cefpodoxime proxetil, cefcapene pivoxil, amoxicillin, temocillin, a pharmaceutically acceptable salt thereof and the like.

Examples of the antiglaucoma agent include carteolol, timolol, latanoprost, travoprost, tafluprost, unoprostone, betaxolol, befunolol, levobunolol, nipradilol, dipivefrin, epinephrine, acetazolamide, brinzolamide, dorzolamide, a pharmaceutically acceptable salt thereof and the like.

The vasoconstrictor in the present invention need only have a blood vessel contracting action, particularly one showing such action by transdermal administration, and phenylephrine, naphazoline, ephedrine, methylephedrine, tetetrahydrozoline, epinephrine, norepinephrine, pseudoephedrine, etilefrine, dopamine, a pharmaceutically acceptable salt thereof and the like can be mentioned. Preferred are phenylephrine hydrochloride, naphazoline hydrochloride, ephedrine hydrochloride, epinephrine and tetrahydrozoline hydrochloride, and more preferred is phenylephrine hydrochloride.

The preparation of the present invention containing an ophthalmic drug and a vasoconstrictor need only be in the form capable of increasing the transfer amount of an ophthalmic drug (e.g., an antiallergic agent) through the eyelid skin into a topical area in the eye, particularly an anterior segment of the eye, by administration of the preparation to the skin surface of an eyelid. For example, an external preparation such as adhesive preparation, ointment, gel preparation, cream and the like can be mentioned. Preferred is adhesive preparation and gel preparation. In the present invention, moreover, the adhesive preparation means a preparation that can be adhered to the skin such as cataplasm, patch, tape preparation, plaster and the like. In the case of two kinds of preparations obtained by separately formulating an ophthalmic drug and a vasoconstrictor, they may have the same form or different forms.

In the present invention, the “skin surface of an eyelid” means the upper eyelid, the lower eyelid and the skin surface in the vicinity thereof.

In the present invention, the “topical area in the eye” means an eye tissue including the anterior segment of the eye.

In the present invention, the “anterior segment of the eye” refers to conjunctiva, lacrimal fluid, aqueous humor and cornea.

The preparation of the present invention permits an increase in the transfer amount of an ophthalmic drug into the anterior segment of the eye by controlling the kind, amount and the like of the ophthalmic drug and the vasoconstrictor to be contained in the preparation.

Where necessary, the preparation of the present invention can appropriately contain, as additive, any component generally used for the production of pharmaceutical products, as long as the effect of the invention is not impaired. For example, gel base, a base for matrix type adhesive preparation, ointment base, solvent, oil solution, surfactant, gum, resin, absorption promoter, wetting agent, buffer, pH adjusting agent and the like can be mentioned.

Examples of the gel base include polymer thickeners such as hydroxypropylmethylcellulose, carboxyvinyl polymer, polyacrylic acid, sodium polyacrylate, methylcellulose, polyvinyl alcohol, polyvinylpyrrolidone, polyethylene oxide, polyacrylamide, sodium alginate, gelatin, gum arabic, gum tragacanth, guar gum, xanthan gum, agar, carageenan, chitosan and the like; fatty acid esters such as isopropyl myristate, isopropyl palmitate, propylene glycol oleate and the like; fatty acids such as lactic acid, lauric acid, oleic acid, linoleic acid, linolenic acid and the like; aliphatic alcohols such as lauryl alcohol, oleyl alcohol and the like; hydrocarbons such as squalene, squalane and the like, and the like.

Examples of the base for matrix type adhesive preparation include acrylic adhesive, silicon adhesive, rubber adhesive and the like, from which the base can be appropriately selected for use. In addition, the matrix type adhesive preparation may be carried on one surface of a support generally used for a preparation to be adhered to the skin such as tape preparation, patch, cataplasm, plaster and the like or a support made of a material free of inconvenience for the use in the present invention, and used.

Examples of the acrylic adhesive include acrylic acid-acrylic acid octyl ester copolymer, acrylic acid ester-vinyl acetate copolymer, acrylic acid 2-ethylhexyl-vinylpyrrolidone copolymer, methacrylic acid-butyl acrylate copolymer and the like.

Examples of the silicon adhesive include polymethylphenylsiloxane copolymer, acrylic acid-dimethylsiloxane copolymer and the like.

Examples of the rubber adhesive include styrene-isoprene-styrene copolymer, styrene-isoprene-styrene block copolymer, natural rubber, polyisobutylene, polybutene, ethylene-vinyl acetate copolymer (EVA) and the like, which are added, where necessary, with tackifier resin, softener and the like, and the like.

Examples of the ointment base include grease base such as petrolatum, paraffin, plastibase, silicone, vegetable oil, lard, wax, simple ointment and the like; emulsion base such as hydrophilic ointment (vanishing cream), hydrophilic petrolatum, purified lanolin, absorption ointment, hydrous lanolin, hydrophilic plastibase (cold cream) and the like, and the like.

Examples of the solvent include purified water, ethanol, lower alcohol, ethers, pyrrolidones, ethyl acetate and the like.

Examples of the oil solution include volatile or nonvolatile oil solution, solvent, resin and the like generally used for skin external preparation, which may be liquid, paste or solid at ambient temperature. For example, higher alcohol such as cetyl alcohol, isostearyl alcohol and the like; fatty acid such as isostearic acid, oleic acid and the like; polyvalent alcohol such as glycerol, sorbitol, ethylene glycol, propylene glycol, polyethylene glycol and the like; esters such as myristyl myristate, hexyl laurate, decyl oleate, isopropyl myristate, glycerol monostearate and the like and the like can be mentioned.

As the surfactant, anionic surfactant, cationic surfactant, nonionic surfactant or amphoteric surfactant can be used.

Examples of the anionic surfactant include fatty acid salt, alkyl sulfate, polyoxyethylene alkyl sulfate, alkyl sulfocarboxylate, alkyl ether carboxylate and the like.

Examples of the cationic surfactant include amine salt, quaternary ammonium salt and the like.

Examples of the nonionic surfactant include polyoxyethylene hydrogenated castor oil, polyoxyethylene fatty acid ester, polyoxyethylene alkyl ether, sorbitan polyoxyethylene fatty acid ester and the like.

Examples of the amphoteric surfactant include alkyl betaine, dimethylalkylglycine, lecithin and the like.

Examples of the gum and resin include cation polymer such as sodium polyacrylate, cellulose ether, calcium alginate, carboxyvinyl polymer, ethylene-acrylic acid copolymer, vinylpyrrolidone polymer, vinyl alcohol-vinyl pyrrolidone copolymer, nitrogen substitution acrylamide polymer, polyacrylamide, cation guar gum and the like, acrylic copolymers such as dimethylacrylic ammonium polymer, acrylic acid methacrylic acid acrylic copolymer and the like, polyoxyethylene-polypropylene copolymer, polyvinyl alcohol, pullulan, agar, gelatin, tamarind seed polysaccharides, xanthan gum, carageenan, chitosan, high methoxylpectin, low methoxylpectin, guar gum, gum arabic, crystalline cellulose, arabino galactan, karaya gum, gum tragacanth, alginic acid, albumin, casein, curdlan, gellan gum, dextran, cellulose, polyethylenimine, high polymerization polyethylene glycol, cation silicone polymer, synthetic latex, acrylic silicone, trimethyl siloxy silicate, fluorinated silicone resin and the like.

Examples of the absorption promoter include 1-dodecylazacycloheptan-2-on, pyrrothiodecane, oleyl alcohol, lauric acid, oleic acid, sodium lauryl sulfate, d-limonene, 1-menthol, 2-pyrrolidone, 1-methyl-2-pyrrolidone, N,N-dimethylformamide, N,N-dimethylacetamide, dimethyl sulfoxide, decylmethyl sulfoxide, N-lauroylsarcosine, isopropyl myristate, isopropyl palmitate, fumaric acid, maleic acid, sorbic acid, glycyrrhizinic acid, myristyl lactate, cetyl lactate, polyoxyethyleneoleyl ether, lauric acid diethanolamide, polyvalent alcohols, glycerol, propylene glycol, diethanolamine, triisopropanolamine, triethanolamine and the like, which may be used in a combination of two or more kinds thereof. Preferred is isopropyl myristate.

Examples of the wetting agent include glycerol, polyethylene glycol, sorbitol, maltitol, propylene glycol, 1,3-butanediol, hydrogenated maltose starch syrup and the like.

Examples of the buffer include phosphoric acid or a salt thereof (phosphoric acid dihydrogen sodium, phosphoric acid monohydrogen sodium etc.), boric acid or a salt thereof (borax etc.), acetic acid or a salt thereof (sodium acetate etc.), citric acid or a salt thereof (sodium citrate etc.), amino acid such as glutamic acid or epsilon aminocaproic acid, carbonate buffer, Tris buffer and the like, and a combination thereof.

Examples of the pH adjusting agent include sodium hydroxide, potassium hydroxide, sodium carbonate, hydrochloric acid, phosphoric acid, acetic acid, citric acid and the like.

The preparation of the present invention can be produced according to a conventional method. In the case of a gel preparation, for example, it can be produced by adding a solvent to a gel base, neutralizing the mixture by adding a pH adjusting agent, blending, where necessary, the mixture with a solvent, an oil solution, a surfactant, a gum, a resin, an absorption promoter, a wetting agent, a buffer and the like, adding an ophthalmic drug and a vasoconstrictor thereto and thoroughly kneading the mixture.

In the case of an adhesive preparation (cataplasm, patch, tape preparation, plaster), it can be produced by adding a base of matrix type preparation and/or gum and, where necessary, a solvent, an oil solution, a surfactant, a resin, an absorption promoter, a wetting agent and the like to an ophthalmic drug and a vasoconstrictor, thoroughly mixing them, spreading the plaster on a support such as a non-woven fabric, a woven fabric, a plastic film (including sheet), a film made of a composite thereof, and the like, covering the product with a release liner, or spreading the plaster on a release liner, and pressure transferring the product onto the aforementioned support.

It is preferable that the aforementioned support should have flexibility allowing adhesion thereof to the skin surface of an eyelid. While the thickness is appropriately determined according to the dosage form, in consideration of the strength of the preparation and foreign body sensation and adhesiveness during adhesion, it is preferably within the range of 10-3000 μm.

In the case of an ointment, it can be produced by adding an ophthalmic drug and a vasoconstrictor, and an ointment base and, where necessary, a solvent, an oil solution, a surfactant, gum, a resin, an absorption promoter, a wetting agent and the like and thoroughly mixing them.

The preparation of the present invention can contain, in addition to the above-mentioned components, stabilizer, antioxidant, preservative, crosslinking agent, pH adjusting agent, UV absorber and the like, as long as the effect of the invention is not impaired.

The content of the ophthalmic drug in the preparation of the present invention is generally 0.01-40 wt %, preferably 0.1-30 wt %, and particularly preferably 0.5-20 wt %. For example, when an antiallergic agent is used as ophthalmic drug, a content of antiallergic agent is generally 0.01-40 wt %, preferably 0.1-30 wt %, and particularly preferably 0.5-20 wt %.

When the preparation of the present invention is used as an adhesive preparation, for example, the content of the antiallergic agent is preferably within the range of 0.01-40 wt %, more preferably 0.1-30 wt %, and particularly preferably 0.5-20 wt %. When it is used as an ointment or gel preparation, for example, the content of the antiallergic agent is preferably within the range of 0.01-40 wt %, more preferably 0.1-30 wt %, and particularly preferably 0.5-20 wt %.

The content of the vasoconstrictor in the preparation of the present invention is generally 0.001-30 wt %, preferably 0.01-20 wt %, and particularly preferably 0.1-10 wt %.

When the preparation of the present invention is used as adhesive preparation, a content of vasoconstrictor is preferably within the range of 0.001-30 wt %, more preferably 0.01-20 wt %, and particularly preferably 0.1-10 wt %. In the case of being used as ointment or gel preparation, a content of vasoconstrictor is preferably within the range of 0.001-30 wt %, more preferably 0.01-20 wt %, and particularly preferably 0.1-10 wt %.

In addition, as long as the object of the present invention is achieved, the preparation of the present invention may be formulated into a preparation containing a pharmaceutical ingredient other than the antiallergic agent, such as steroid or non-steroidal anti-inflammatory agent, antivirus agent, mydriatic drug, anticholinesterase agent, miotic drug, antibiotic, sulfa drug, surface anesthetic, vitamins and the like.

In the preparation of the present invention, while the dose of the ophthalmic drug varies depending on the pathology and age of patient, administration form and the like, in the case of, for example, an antiallergic agent, the daily dose for an adult is generally about 0.01 mg-500 mg/day, preferably about 0.05 mg-50 mg/day, more preferably about 0.1 mg-10 mg/day, which is administered in 1 to 5 portions as necessary. In addition, the preparation of the present invention can be administered during sleep.

In the preparation of the present invention, the dose of the vasoconstrictor varies depending on the pathology and age of patient, administration form and the like, the daily dose for an adult is generally about 0.001 mg-200 mg/day, preferably about 0.01 mg-50 mg/day, more preferably about 1 mg-10 mg/day, which is administered in 1 to 5 portions as necessary.

By administration of the preparation of the present invention to the skin surface of an eyelid, the transfer amount of an ophthalmic drug into a topical area in the eye, particularly the anterior segment of the eye, cornea and the like, through the eyelid skin can be increased. Therefore, the preparation is useful as an agent for the prophylaxis or treatment of a disease in the anterior segment of the eye.

The preparation of the present invention can exert an antiallergic effect in a sustained manner, and therefore, is useful as an agent for the prophylaxis or treatment of allergic diseases. Examples of the allergic disease include allergic conjunctivitis, vernal keratoconjunctivitis, contact eyelid conjunctivitis, phlyctenular keratoconjunctivitis, giant papillary conjunctivitis and the like.

The subject of administration of the preparation of the present invention is not particularly limited, and various mammals such as human, monkey, mouse, rat, guinea pig, rabbit, swine, dog, horse, bovine and the like can be mentioned. The preparation of the present invention is useful for allergic disease and the like in the aforementioned animals.

The present invention provides a method of increasing the amount of transfer of an ophthalmic drug to a topical area in the eye through the eyelid, which comprises a step of administering an ophthalmic drug to the skin surface of an eyelid under the conditions where a vasoconstrictor is present from the skin surface of an eyelid to the inside of the eyelid and/or conjunctiva.

For the application of the method, a method similar to the one explained with regard to the aforementioned combined use can be employed.

The present invention also provides a method of treating an ophthalmic disease, which comprises a step of administering an effective amount of an ophthalmic drug and a vasoconstrictor to a subject of administration in need of the treatment. As the ophthalmic disease, allergic disease and the like can be mentioned.

The present invention also provides use of an ophthalmic drug and a vasoconstrictor for the production of an ophthalmic percutaneous absorption type preparation containing an ophthalmic drug and a vasoconstrictor.

Furthermore, the present invention provides use of an ophthalmic drug and a vasoconstrictor for the production of an ophthalmic percutaneous absorption type preparation.

The present invention provides a commercial package comprising the ophthalmic percutaneous absorption type preparation of the present invention containing an antiallergic agent as an ophthalmic drug, and a written matter stating that the preparation can or should be used for the prophylaxis or treatment of an allergic disease.

As the aforementioned written matter, what is called a package insert that describes an explanation relating to use, efficacy, administration method and the like, and the like can be mentioned.

### EXAMPLES

While the present invention is explained in more detail by referring to the following Experimental Examples and Examples, which are not to be construed as limitative.

### Test Method and Results

### <Experiment Material 1>

ketotifen fumarate (Sigma Ltd.), phenylephrine hydrochloride (biochemical use, Wako Pure Chemical Industries, Ltd.), hydroxypropylmethylcellulose (Metolose 60SH-4000, Shin-Etsu Chemical Co., Ltd.), sodium dihydrogenphosphate dihydrate (primary, Wako Pure Chemical Industries, Ltd.) and sodium hydroxide (The Japanese Pharmacopoeia, Nacalai Tesque)

### <Test Preparation 1>

### Comparative Example 1

### Gel Preparation Containing 20% Ketotifen Fumarate

### Example 1

### Gel Preparation Containing 20% Ketotifen Fumarate and 2% phenylephrine hydrochloride

### Example 2

### Gel Preparation Containing 20% Ketotifen Fumarate and 4% Phenylephrine Hydrochloride

These preparations were produced according to the formulation of Table 1 and the below-mentioned preparation method.

TABLE 1

| Component | Com. Ex. 1 | Ex. 1 | Ex. 2 |
|---|---|---|---|
| ketotifen fumarate | 20 | 20 | 20 |
| phenylephrine hydrochloride | — | 2 | 4 |
| hydroxypropylmethylcellulose | 3 | 3 | 3 |
| sodium dihydrogenphosphate | 0.156 | 0.156 | 0.156 |
| dihydrate | | | |
| purified water | adequate | adequate | adequate |
| | dose | dose | dose |
| sodium hydroxide | adequate | adequate | adequate |
| | dose | dose | dose |
| pH | 6 | 6 | 6 |
| total | 100 | 100 | 100 |

(unit: w/w %)

### <Preparation Method 1>

### Comparative Example 1 Preparation

Sodium dihydrogenphosphate dihydrate was added to purified water and the mixture was stirred until complete dissolution. The solution was heated in a water bath heated to approximately 70° C., hydroxypropylmethylcellulose was added by small portions and dissolved with stirring. This was left standing for 10 min at room temperature, and 1N aqueous sodium hydroxide solution was added. The mixture was adjusted to pH 6 to give a gel base. Ketotifen fumarate and the gel base were measured on a glass petri dish, and sufficiently stirred with a spatel to give the Comparative Example 1 preparation (gel preparation containing 20% ketotifen fumarate).

### Example 1 Preparation and Example 2 Preparation

Sodium dihydrogenphosphate dihydrate and phenylephrine hydrochloride were added to purified water and the mixture was stirred until complete dissolution. The solution was heated in a water bath heated to approximately 70° C., hydroxypropylmethylcellulose was added by small portions and dissolved with stirring. This was left standing for 10 min at room temperature, and 1N aqueous sodium hydroxide solution was added. The mixture was adjusted to pH 6 to give a phenylephrine hydrochloride-containing gel base. Ketotifen fumarate and the phenylephrine hydrochloride-containing gel base were measured on a glass petri dish, and sufficiently stirred with a spatel to give the Example 1 preparation (2% phenylephrine hydrochloride and 20% ketotifen fumarate-containing gel preparation) and the Example 2 preparation (4% phenylephrine hydrochloride and 20% ketotifen fumarate-containing gel preparataion).

### <Animal Used 1>

Male Japanese white rabbits (purchased from KITAYAMA LABES Co., Ltd., body weight 2.2-2.5 kg) were used.

### <Test Method 1>

### 1) Pretreatment of Animal

For administration of the test preparation, the areas surrounding the eyes of rabbit were shaved in advance. The shaving treatment was performed one day before the test under a ketamine/xylazine combined anesthesia using an electric clipper and a shaver with much care not to hurt the skin. An adhesive tape (TC-18, NICHIBAN) was adhered to and detached from the lower eyelid skin 20 times to remove the stratum corneum layer.

### 2) Administration of Test Preparation

A test preparation formed in 2 cm×1 cm×0.108 cm (width×length×thickness, 0.216 cm⁷) on a plastic wrap (Saran Wrap (registered trade mark), Asahi Kasei Corporation) was administered to the lower eyelid skin. To prevent drying of the test preparation, the applied test preparation was covered with the plastic wrap.

### 3) Collection of Eye Tissue

The test preparation was removed 2 hr after administration, and lacrimal fluid was collected by capillary. The rabbit was euthanized with an excess amount of pentobarbital sodium solution, the anterior segment of the eye was washed with saline. The aqueous humor was collected and the eyeball with the conjunctiva was isolated. The conjunctiva was obtained from the isolated eye on a glass petri dish. Then, the eyelid skin was isolated. The non-administration eye was similarly processed to give an eye tissue.

### 4) Pretreatment

conjunctiva: To the collected conjunctiva was added 10 mM sodium dihydrogenphosphate dihydrate buffer (pH 7, 1 mL) to chop the conjunctiva. Acetonitrile (4 mL) was added and the mixture was shaken up and down at 300 rpm for 10 min, and the mixture was centrifuged at 3000 rpm for 10 min. Then, the supernatant (4 mL) was placed in a different test tube, dried under reduced pressure with heating, and dissolved in 300 μL of HPLC mobile phase (having formulation described in the following 5)). Then, the solution was centrifuged at 14000 rpm for 5 min, and the supernatant was used as an HPLC measurement sample.

lacrimal fluid: HPLC mobile phase (200 μL) was added to the collected lacrimal fluid, and the mixture was stirred and centrifuged at 14000 rpm for 5 min. The supernatant was used as an HPLC measurement sample.

aqueous humor: The collected aqueous humor was centrifuged at 14000 rpm for 5 min and the supernatant was used as an HPLC measurement sample.

eyelid skin: (non-administration eye) To the collected eyelid skin was added 10 mM sodium dihydrogenphosphate dihydrate buffer (pH 7, 1 mL) to chop the eyelid skin. Acetonitrile (4 mL) was added and the mixture was shaken up and down at 300 rpm for 10 min, and the mixture was centrifuged at 3000 rpm for 10 min. Then, the supernatant (4 mL) was placed in a different test tube, dried under reduced pressure with heating, and dissolved in 300 μL of HPLC mobile phase. Then, the solution was centrifuged at 14000 rpm for 5 min, and the supernatant was used as an HPLC measurement sample.

(administration eye) To the collected eyelid skin was added 10 mM sodium dihydrogenphosphate dihydrate buffer (pH 7, 1 mL) to chop the eyelid skin. Acetonitrile (4 mL) was added and the mixture was shaken up and down at 300 rpm for 10 min, and the mixture was centrifuged at 3000 rpm for 10 min. Then, the supernatant was diluted 10-fold with the HPLC mobile phase to give an HPLC measurement sample.
 HPLC mobile phase: 0.1M tris(hydroxymethyl)aminomethane buffer (pH 9):acetonitrile=30:70 (v/v %)

### 5) Measurement of Concentration

Using a high performance liquid chromatography, the ketotifen concentration was measured under the following HPLC conditions:

### <HPLC Conditions>

detector: ultraviolet spectrophotometric detector (measurement wavelength 300 nm)
column: Capcell pak C18 MG S5 μm, 4.5×250 mm, Shiseido Co., Ltd.
guard column (TOSOH, ODS-80Ts)
   column temperature: constant temperature near 40° C.
   mobile phase: 0.1M tris(hydroxymethyl)aminomethane buffer (pH 9):acetonitrile=30:70 (v/v %)
   flow rate: 1.0 mL/min
   injection volume: 50 μL

### <Test Results 1>

TABLE 2

| | | Ketotifen concentration | |
|---|---|---|---|
| | | | non- |
| | | administration | administration |
| tissue | preparation | eye | eye |
| eyelid skin | Com. Ex. 1 | 1112 ± 393 | 2 ± 1 |
| [μg/g] | Ex. 1 | 1272 ± 146 | 1 ± 1 |
| | Ex. 2 | 1036 ± 187 | 1 ± 0 |
| conjunctiva | Com. Ex. 1 | 2104 ± 1104 | 1060 ± 390 |
| [ng/g] | Ex. 1 | 4656 ± 5250 | 1241 ± 561 |
| | Ex. 2 | 3414 ± 426 | 1133 ± 98 |
| lacrimal fluid | Com. Ex. 1 | 3313 ± 1834 | 1205 ± 333 |
| [ng/mL] | Ex. 1 | 3800 ± 2249 | 950 ± 297 |
| | Ex. 2 | 39180 ± 17389 | 1719 ± 272 |
| aqueous humor | Com. Ex. 1 | 24 ± 11 | 13 ± 11 |
| [ng/mL] | Ex. 1 | 16 ± 5 | 16 ± 6 |
| | Ex. 2 | 40 ± 36 | 18 ± 1 |

(Each value shows mean ± standard deviation. n = 3.)

As is clear from Table 2, the Example 1 and Example 2 administration groups containing phenylephrine hydrochloride showed an increase in the transfer amount of ketotifen to the conjunctiva, lacrimal fluid and aqueous humor, as compared to the phenylephrine hydrochloride non-addition Comparative Example administration group.

### <Experiment Material 2>

The same materials as in experiment material 1 were used except that ketotifen fumarate was changed to olopatadine hydrochloride. As olopatadine hydrochloride, an extract of our own company was used.

### <Test Preparation 2>

### Comparative Example 2

### Gel Preparation Containing 20% Olopatadine Hydrochloride

### Example 3

### Gel Preparation Containing 20% Olopatadine Hydrochloride and 4% Phenylephrine Hydrochloride

These preparations were produced according to the formulation of Table 3 and the below-mentioned preparation method.

TABLE 3

| component | Com. Ex. 2 | Ex. 3 |
|---|---|---|
| olopatadine hydrochloride | 20 | 20 |
| phenylephrine hydrochloride | — | 4 |
| hydroxypropylmethylcellulose | 3 | 3 |
| sodium dihydrogenphosphate | 0.156 | 0.156 |
| dihydrate | | |
| sodium hydroxide | adequate | adequate |
| | dose | dose |
| purified water | adequate | adequate |
| | dose | dose |
| pH | 6 | 6 |
| total | 100 | 100 |

(unit: w/w %)

### <Extraction-Purification Method of Olopatadine Hydrochloride>

(1) “Allelock® tablets 5”, 1500 tablets (about 187 g), were finely pulverized in a mill.
(2) The ground product was suspended in a mixture of ethanol (500 mL)/1N sodium hydroxide aqueous solution (20 mL), vigorously stirred at room temperature for about 1 hr, and the insoluble material was collected by filtration. Then, ethanol (500 mL) was added to the insoluble material, and the mixture was vigorously stirred at room temperature for about 1 hr and filtrated to give the insoluble material again. This operation was repeated twice.
(3) The obtained filtrate (about 2 L) was concentrated to about 100 mL. To this solution was added purified water (about 900 mL) to give about 1 L of a suspension. This was filtrated to give a filtrate (pH 5-6).
(4) The filtrate (about 1 L) was passed through DIAION HP-20 (500 mL) to allow adsorption. The resin was desalted by washing with purified water (about 1 L). Thereafter, the resin was washed twice with 20, 40, 60% (v/v) methanol aqueous solution (500 mL) and eluted with methanol (about 2.3 L). The fraction (about 2 L) showing a single spot was concentrated to give a mixture of olopatadine free forms (about 7.8 g).
(5) The obtained olopatadine free forms (about 5.9 g) were recrystallized from 2-propanol/purified water mixed solution (3:1, about 100 mL).
(6) The crystals of the free form were dissolved in 2-propanol/purified water mixed solution (3:1, about 50 mL)/methanol (about 10 mL) mixed solution, and the solution was concentrated for several-min to evaporate methanol in the filtrate. 4N HCl/dioxane (4.25 mL, 1 eq) was added to the solution and the mixture was cooled or concentrated to give crystals.
(7) The obtained crystals were filtrated under reduced pressure and, after evaporating the redundant solvent, dried under reduced pressure at room temperature for about 20 hr to give olopatadine hydrochloride as a white powder (about 4.1 g: yield 54.7%). The chemical structure, property and purity of the obtained olopatadine hydrochloride were confirmed by nuclear magnetic resonance spectrum (¹H-NMR), melting point measurement, water content measurement, and high performance liquid chromatography (HPLC).

### <Preparation Method 2>

The preparation method was the same as preparation method 1 except that olopatadine hydrochloride was used instead of ketotifen fumarate.

### <Animal Used 2>

Male Japanese white rabbits (purchased from KITAYAMA LABES Co., Ltd., body weight 2.4-2.6 kg) were used.

### <Test Method 2>

### 1) Pretreatment of Animal

Same as in the above-mentioned test method 1.

### 2) Test Preparation Administration

Same as in the above-mentioned test method 1.

### 3) Collection of Lacrimal Fluid-Blood

At 2 hr after the administration, the test preparation was removed and the lacrimal fluid was collected by capillary. Thereafter, the blood was drawn from the heart.

### 4) Pretreatment

lacrimal fluid: LC/MS/MS mobile phase (200 μL) was added to the collected lacrimal fluid, and the mixture was stirred and centrifuged at 14000 rpm for 5 min. The supernatant was collected, filtrated with an aqueous-non-aqueous filter (4P, 0.45 μm, GL Sciences, Inc.), and the filtrate was used as an LC/MS/MS measurement sample.
blood: The collected blood was centrifuged (TOMY, HF-120) to give plasma. Purified water (1 mL) was added to the plasma (1 mL) and the mixture was sufficiently stirred. The solution was passed through a pretreated column (pretreatment: 1% formic acid containing methanol (1 mL×1) and purified water (1 mL×2),
column: BOND ELUT-C18, 50 MG, 1 mL). The column was washed (purified water was passed (1 mL×2)), and 1% formic acid containing methanol (1 mL×2) was passed through the column to elute the drug (eluate). The recovered eluate was concentrated by spraying nitrogen thereon, and dissolved in LC/MS/MS mobile phase (300 μL). After filtration using a filter (4P, 0.45 μm, GL Sciences, Inc.), the filtrate was diluted 10-fold with the mobile phase and used as an LC/MS/MS measurement sample.
LC/MS/MS mobile phase: 10 mM acetic acid
solution:methanol=55:45 (v/v %)

### 5) Measurement of Concentration

The olopatadine concentration was measured using the LC/MS/MS system under the following conditions.

LC/MS/MS system
MS/MS part: API-4000 (Applied Biosystems)
nitrogen/Zero Air development apparatus (KN-2-20016, Kaken Geneqs Inc.)
vacuum pump (HS-602, VARIAN)
oil-free scroll compressor (SLP-151CD-S1, ANEST IWATA Corporation)
LC part: NANOSPACE SI-2 series (Shiseido Co., Ltd.):
pump 1 (NANOSPACE SI-2 3001)
pump 2 (NANOSPACE SI-2 3001)

### Degasser (NANOSPACE SI-2 3009)

UV detector (NANOSPACE SI-2 3002)

### Column Oven (NANOSPACE SI-2 3004)

autoinjector (NANOSPACE SI-2 3133)

### LC Conditions

column: Capcell pak C18 MGII S-5 μm, 1.5×75 mm, Shiseido Co., Ltd.
column temperature: constant temperature near 40° C.
mobile phase: pump 1: methanol
pump 2: 10 mM acetic acid solution
flow rate: pump 1: 45 μL/min
pump 2: 55 μL/min
injection volume: 5 μL

The MS/MS conditions are as shown in Table 4.

TABLE 4

| | MS/MS condition |
|---|---|
| Scan Type | MRM(MRM) |
| Polarity | Positive |
| Ion source | Turbo spray (ESI) |
| Cur: Curtain Gas (psi) | 40.00 |
| GS1: Ion Source Gas 1 (psi) | 70.00 |
| GS2: Ion Source Gas 2 (psi) | 70.00 |
| IS: Ion Spray Voltage (V) | 5500.00 |
| TEM: Temperature (° C.) | 600.00 |
| ihe: Interface Heater | ON |
| CAD: Collision Gas | 7.00 |
| DP: Declistering Potential (V) | 76.00 |
| EP: Entrance Potential (V) | 10.00 |
| CE: Collision Energy (V) | 33.00 |
| CXP: Collision Cell Exit | 10.00 |
| Potential (V) | |
| Monitor ion (Q1→Q3) | 338.30→165.10 |
| Acquisition (min) | 6 |

Parameters in the Analyst ® software of Applied Biosystems are shown.

### <Test Results 2>

TABLE 5

Concentration of olopatadine in tissue [ng/mL]

| preparation | lacrimal fluid | plasma |
|---|---|---|
| Ex. 3 | 2436 ± 3650 | 689 ± 207 |
| Com. Ex. 2 | 388 ± 603 | 1124 ± 157 |

(Each value shows mean ± standard deviation. n = 3.)

As is clear from Table 5, the Example 3 administration group using phenylephrine hydrochloride showed a decreased amount of olopatadine transferred to the plasma but an increased amount thereof to the lacrimal fluid, as compared to the Comparative Example 2 administration group free of phenylephrine hydrochloride addition.

### Formulation Example 1

| ketotifen fumarate | 0.3 | g |
|---|---|---|
| phenylephrine hydrochloride | 0.12 | g |
| isopropyl myristate | 1.2 | g |
| acrylic copolymer | 1.295 | g |
| polyisocyanate compound | 0.0015 | g |
| ethyl acetate | adequate dose | |
| total amount | 3 | g |

Ethyl acetate (about 2 mL) is added to and mixed with ketotifen fumarate and phenylephrine hydrochloride, and the mixture is sonicated in a disposable cup for about 30 sec to dissolve or disperse ketotifen fumarate and phenylephrine hydrochloride. Isopropyl myristate is added and the mixture is sufficiently mixed. Then, an acrylic copolymer acrylic adhesive as an adhesive base and a polyisocyanate compound as a crosslinking agent are successively added and the mixture is sufficiently mixed. The mixture is deaerated, spread on a release liner with a doctor knife or Baker applicator, and stood still until the organic solvent is evaporated. Then, a support is applied thereon and pressure bonded with a roller, which is followed by crosslinking in a thermostatic tank at about 40° C. for 8-12 hr to give a phenylephrine hydrochloride-ketotifen fumarate-containing tape preparation.

### Formulation Example 2

| | | |
|---|---|---|
| olopatadine hydrochloride | 0.3 | g |
| naphazoline hydrochloride | 0.06 | g |
| isopropyl myristate | 1.2 | g |
| white petrolatum | 1.44 | g |
| total amount | 3 | g |

White petrolatum and isopropyl myristate are thoroughly mixed, olopatadine hydrochloride and naphazoline hydrochloride are added to the mixed ointment base and the mixture is thoroughly kneaded to give a naphazoline hydrochloride-olopatadine hydrochloride-containing ointment.

### Formulation Example 3

| epinastine hydrochloride | 0.3 | g |
|---|---|---|
| ephedrine hydrochloride | 0.12 | g |
| sodium polyacrylate | 0.45 | g |
| glycerol | 0.3 | g |
| peppermint oil | 0.01 | g |
| purified water | adequate dose | |
| total amount | 3 | g |

Purified water is thoroughly mixed with sodium polyacrylate and glycerol to give a water-containing plaster.

Furthermore, peppermint oil, epinastine hydrochloride and ephedrine hydrochloride are added and the mixture is thoroughly kneaded. The plaster mixture is spread and formed on a support (polyester non-woven fabric etc.), and a release liner is applied to give an ephedrine hydrochloride-epinastine hydrochloride-containing cataplasm.

While some of the embodiments of the present invention have been described in detail in the above, it is, however, possible for those of ordinary skill in the art to make various modifications and changes to the particular embodiments shown without substantially departing from the teaching and advantages of the present invention. Such modifications and changes are encompassed in the spirit and scope of the present invention as set forth in the appended claims.

This application is based on U.S. provisional application 60/840,462, the contents of which are incorporated in full herein by this reference.

1. An ophthalmic percutaneous absorption type preparation comprising an ophthalmic drug and a vasoconstrictor in combination.

2. An ophthalmic percutaneous absorption type preparation comprising an ophthalmic drug and a vasoconstrictor.

3. The ophthalmic percutaneous absorption type preparation of claim 1, which is administered to the skin surface of an eyelid.

4. The ophthalmic percutaneous absorption type preparation of claim 1, wherein the ophthalmic drug is an agent for the prophylaxis or treatment of a disease in the anterior segment of the eye.

5. The ophthalmic percutaneous absorption type preparation of claim 4 wherein the agent for the prophylaxis or treatment of a disease in the anterior segment of the eye is at least one selected from an antiallergic agent, a therapeutic agent for dry eye, an anti-inflammatory agent, an antibacterial agent and an antiglaucoma agent.

6. The ophthalmic percutaneous absorption type preparation of claim 5, wherein the agent for the prophylaxis or treatment of a disease in the anterior segment of the eye is an antiallergic agent.

7. The ophthalmic percutaneous absorption type preparation of claim 6, wherein the antiallergic agent is at least one selected from ketotifen, olopatadine, epinastine and a pharmaceutically acceptable salt thereof.

8. The ophthalmic percutaneous absorption type preparation of claim 7, wherein the antiallergic agent is ketotifen fumarate or olopatadine hydrochloride.

9. The ophthalmic percutaneous absorption type preparation of claim 8, wherein the vasoconstrictor is phenylephrine hydrochloride.

10. A method of increasing the amount of transfer of an ophthalmic drug to a topical area in the eye through the eyelid, which comprises a step of administering an ophthalmic drug to the skin surface of an eyelid under the conditions where a vasoconstrictor is present from the skin surface of an eyelid to the inside of the eyelid and/or conjunctiva.

11. The method of claim 10, wherein the topical area in the eye is the anterior segment of the eye.

12. The method of claim 10, wherein the ophthalmic drug is an agent for the prophylaxis or treatment of a disease in the anterior segment of the eye.

13. The method of claim 12, wherein the agent for the prophylaxis or treatment of the disease in the anterior segment of the eye is at least one selected from an antiallergic agent, a therapeutic agent for dry eye, an anti-inflammatory agent, an antibacterial agent and an antiglaucoma agent.

14. The method of claim 13, wherein the agent for the prophylaxis or treatment of the disease in the anterior segment of the eye is antiallergic agent.

15. The method of claim 14, wherein the antiallergic agent is at least one selected from ketotifen, olopatadine, epinastine and a pharmaceutically acceptable salt thereof.

16. The method of claim 15, wherein the antiallergic agent is ketotifen fumarate or olopatadine hydrochloride.

17. The method of claim 16, wherein the vasoconstrictor is phenylephrine hydrochloride.

18. A method of treating an ophthalmic disease, which comprises a step of administering an effective amount of an ophthalmic drug and a vasoconstrictor to a subject of administration in need of the treatment.

19. (canceled)
